(19) United States
(12) Patent Application Publication
(10) Pub. No.: US 2017/0266090 A1
(43) Pub. Date: Sep. 21, 2017

(54) ANTIMICROBIAL COMPOSITIONS THAT ARE DERMATOLOGICALLY NON-DRYING

(71) Applicant: Marvel Technologies USA, LLC, Franklin, TN (US)

(72) Inventor: Jack A. Wheeler, Nashville, TN (US)

(21) Appl. No.: 15/461,118

(22) Filed: Mar. 16, 2017

Related U.S. Application Data
(60) Provisional application No. 62/309,565, filed on Mar. 17, 2016.

(51) Int. Cl. A61K 8/41; A61K 8/37; A61K 8/36; A01N 25/00; A61K 8/64; A61K 8/97; A01N 33/12; A61Q 17/00; A61K 8/35

(52) U.S. Cl. CPC A61K 8/416; A61Q 17/005; A61K 8/37; A61K 8/36; A61K 8/35; A61K 8/64; A61K 8/97; A01N 33/12; A01N 25/00

(57) ABSTRACT

The subject matter described herein is directed to sanitizing compositions comprising: i. an antimicrobial component comprising a mixture of a quaternary ammonium salt, e.g., benzalkonium chloride and/or benzethonium chloride, paraben and acetic acid in a ratio of about 1:0.8-1.3:1.6-2.5 (w:w:v); ii. a lower alkyl ketone; and iii. an emollient; and optionally, iv. a fragrance. Methods of using and making the compositions are described as well as articles of manufacture incorporating the compositions.

### CROSS-REFERENCE TO RELATED APPLICATIONS

This application claims the benefit of United States Provisional Application No. 62/309,565, filed Mar. 17, 2016, the disclosure of which is incorporated herein by reference in its entirety.

### RELATED FIELD

The subject matter described herein relates to compositions that are useful for sanitizing skin.

### BACKGROUND

Microbes such as bacteria, viruses, spores, algae, and fungi are often found on the skin. These microbes are often the cause of disease and their presence on skin can facilitate the spreading of disease. Accordingly, there is a concern for sanitizing, both of person and property. For example, hand sanitizing equipment is now routinely found in many public locations, in particular, facilities associated with either the medical or food industry. It has been recommended that hands should be washed or sanitized prior to the preparation of food or eating; treating wounds, giving medicine, or caring for a sick or injured person; and even prior to inserting or removing contact lenses.

Many products are putative antimicrobial compositions. However, a majority of the hand sanitizing products available in the market are alcohol based products which contain ethanol, often at more than 60 percent alcohol concentration, that is deemed necessary to kill most harmful bacteria and viruses. Alcohol is also often added to the formulation as a drying agent to hasten evaporation of the solution from the skin. However, the use of alcohol has drawbacks, such as drying of the skin. While emollients have been added to attempt to ameliorate this effect, the products can still cause dry and irritated skin. The alcohol content that causes skin dryness will, in time, also lead to broken skin, which exposes the body to the same contagions users are trying to prevent. The high alcohol content also makes this type of sanitizer flammable.

Alcohol-free sanitizing solutions are known, but a drawback with this type of formulation is that a large amount of chemical antimicrobial agents are used as well as other included additives, such as organic solvents, emulsifiers, drying agents, etc. As such, the benefits of the absence of alcohol must be weighed against the presence of many additional chemicals that are being applied to the skin.

So-called natural health care and skin care products that have purported antimicrobial properties are on the market. However, many of the known natural antimicrobial products are associated with disadvantages, such as, for example, having selective activity, i.e., some of these products work well against bacteria, but not against viruses or fungi, while some will work only against certain strains of bacteria. Moreover, many products lose their activity over a period of time as the pathogens become resistant to the product.

What is therefore needed is an antimicrobial solution that has the desired affects without the excessive drying of the skin. The compositions and methods described herein address these shortcomings of the art.

### BRIEF SUMMARY

In one embodiment, the subject matter described herein is directed to a concentrated sanitizing composition comprising: i. an antimicrobial component comprising a mixture of a quaternary ammonium salt, e.g., benzalkonium chloride and/or benzethonium chloride, preferably benzethonium chloride as the sole quaternary ammonium salt, paraben and acetic acid in a ratio of about 1:0.8-1.3:1.6-2.5 (w:w:v), wherein the mixture comprises from about 0.00001% to about 0.1% (w/v); or from about 0.0002% to about 0.05% (w/v); or from about 0.03% to about 0.05% quaternary ammonium salt (w/v); ii. a lower alkyl ketone; and iii. an emollient.

In one embodiment, the subject matter described herein is directed to a package containing a single use article of a concentrated sanitizing composition comprising: i. an antimicrobial component comprising a mixture of quaternary ammonium salt, e.g., benzalkonium chloride and/or benzethonium chloride, preferably benzethonium chloride as the sole quaternary ammonium salt, paraben and acetic acid in a ratio of about 1:0.8-1.3:1.6-2.5 (w:w:v), wherein the mixture comprises from about 0.00001% to about 0.1% (w/v); or from about 0.0002% to about 0.05% (w/v); or from about 0.03% to about 0.05% quaternary ammonium salt (w/v); ii. a lower alkyl ketone; and iii. an emollient.

In one embodiment, the subject matter described herein is directed to methods of sanitizing skin by contacting the area of skin to be sanitized with a concentrated sanitizing composition comprising: i. an antimicrobial component comprising a mixture of quaternary ammonium salt, e.g., benzalkonium chloride and/or benzethonium chloride, preferably benzethonium chloride as the sole quaternary ammonium salt, paraben and acetic acid in a ratio of about 1:0.8-1.3:1.6-2.5 (w:w:v), wherein the mixture comprises from about 0.00001% to about 0.1% (w/v); or from about 0.0002% to about 0.05% (w/v); or from about 0.03% to about 0.05% quaternary ammonium salt chloride (w/v); ii. a lower alkyl ketone; and iii. an emollient, wherein the skin is sanitized.

### DETAILED DESCRIPTION

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims.

Disclosed herein are sanitizing compositions that advantageously provide desirable anti-microbial efficacy on the skin while avoiding excess drying of the skin. Indeed, the formulations described fully herein can be used repeatedly and often without any appreciable drying of the skin. In a study performed in a surgical practice averaging about 40 procedures per day, surgical personnel reported that frequent use of the compositions disclosed herein did not result in drying of the skin. In fact, surgical personnel reported that there was no observable dryness, cracking or tingling of the skin. All of these symptoms were present when the personnel used an alcohol-based sanitizer. Advantageously, for effective sanitization, the compositions only need to be wet on the skin for about 15 to nearly 20 seconds, which correlates well with the drying time of no more than 20 seconds for the compositions. The data for this study are provided below.

Single-use articles and methods of sanitizing skin are also described herein. The experimental results of tests performed on the compositions described herein show that the compositions do not cause drying of the skin. This is particularly advantageous to those who are required to frequently wash their hands, such a medical personnel and those involved in the food industry.

Paradoxically, one of the aspects of the present disclosure that yields the desired products that are non-drying to the skin is the use of a lower alkyl ketone, such as acetone, which is also known to cause dry skin at high concentrations. While acetone present in formulations is not known to be as effective a drying agent as is ethanol, the present inventors have found an “oil effect” of the acetone component in combination with the particular quaternary ammonium salt, e.g., benzalkonium chloride and/or benzethonium chloride, in particular, benzethonium chloride antimicrobial component whereby the composition is essentially dry within about 15 to less than 20 seconds of application, does not leave a sticky residue and does not cause excessive dry skin like the dryness caused by alcohol containing sanitizers. Importantly, acetone has been found to be particularly compatible with the selected anti-microbial component, which is a combination of three ingredients. This unexpected compatibility and utility led to the formulation of the presently disclosed efficacious, yet non-drying sanitizing compositions, which will now be fully described.

Efficacious skin sanitizing can be accomplished in shorter times than for traditional hand sanitizers. For example, it is generally recommended to rub traditional sanitizing solutions on hands for at least about 20 seconds. The high ethanol content of these known sanitizing solutions is purported to kill most microbes in that time, but any less time is generally considered ineffective or much less effective. It is evident that most persons do not watch a clock while washing hands to ensure the full 20 seconds of scrubbing to provide the necessary time for anti-microbial action. What is needed is a solution that is anti-microbial in as short a time as possible. However, the drying time must not be too short, for example, in the case of products containing high concentrations of ethanol. Such products dry in less than about 15 seconds, and it has been found that in order to effectively kill microorganisms on skin, the skin must remain wet for at least 15 seconds. The present compositions provide this needed solution as the kill-time and the drying time both are about 15 to 20 seconds or less.

Convenience is also an important aspect of a successful skin sanitizer and this is not just a matter of convenient packaging. The compositions disclosed herein are advantageously foaming. Accordingly, the solution is plainly visible to ensure that the skin is completely covered. While there are known foaming hand sanitizers, those typically contain foamers and chemical additives to ensure foaming. None of those ingredients are required in the presently disclosed compositions. Accordingly, the elegant solution that provides efficacy and non-drying also provides a product that foams when dispensed from conventional hand sanitizer dispensers.

As an additional benefit, the presently disclosed skin sanitizers are pleasant in feel and smell, unlike many known skin sanitizers. While the foaming action of the compositions adds to the pleasant feel, the relatively mild anti-microbial agent can be optionally masked if desired by a choice of fragrances.

### Definitions

“Microbicidal,” “bactericidal” or “anti-microbial,” as used herein, refers to lethal, irreversible action resulting in efficacious microbial (or bacterial) cell destruction or incapacitation. On the other hand, microbistatic or bacteristatic, as used herein, refers to reversible anti-microbial (or anti-bacterial) properties, such that if the organism is rendered free of the agent, the organism can again multiply. Differentiation of antimicrobial “-cidal” or “-static” activity, describe the degree of efficacy. A sanitizer and a disinfectant are, by definition, agents which provide microbicidal activity.

As used herein, the term microorganism refers to pathogens and organisms such as Staphylococcus, and any such strains, Salmonella, Escherichia, Campylobacter, Listeria, Pseudomonas and Enterobacteriacae.

The term “microbicidally effective” or “bactericidally effective” means that treatment results in at least a two log₁₀ reduction and more preferably a three log₁₀ reduction in the resident microbial preparation. As described herein, a three and a half log₁₀ reduction up to a five log₁₀ reduction in up to 20 seconds is a sanitizing treatment. The compositions described herein provide this level of sanitization in 15 to 20 seconds. Without being bound to theory, the treatment is believed to destroy the bacterial cell wall.

The compositions described herein are effective at destroying over 99.99% of selected pathogenic and potentially pathogenic microorganisms, both gram negative and gram positive, on inanimate and animate surfaces under conditions prescribed by the appropriate government regulatory agency such as the U.S. E.P.A. or Health Canada. The term “antibacterial” is used to mean capable of destroying microorganisms, but less than 99.9%, within a period of time. The term “fungicidal” is used to mean capable of destroying over 99.99% of selected fungi, particularly Trichophyton mentagrophytes, including their spores within a period of time. The term “virucidal” is used to mean capable of destroying over 99.99% of selected viruses within a period of time. The term “germicidal” is used to mean capable of destroying pathogenic and potentially pathogenic microorganisms. The term “bactericidal” is used to mean capable of destroying bacteria, but not necessarily bacterial spores or mycobacteria.

The term “essentially-free of microbial contamination,” as used herein, means that no microbial contamination can be detected using standard, industry-accepted procedures, such as the AOAC method or simply by swabbing a surface, i.e., skin, to be tested followed by swiping the same swab across an agar plate and subsequently checking for microbial growth.

The term “paraben” refers to a parahydroxybenzoic acid ester. Most preferably, the paraben of the present invention is an alkyl paraben having an alkyl group of from 1-4 carbon atoms, e.g., methyl paraben, ethyl paraben, propyl paraben or butyl paraben.

The term “benzethonium chloride” refers to a quaternary ammonium salt having the structure:

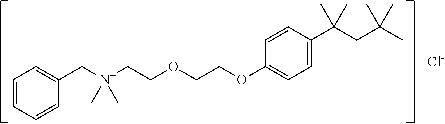

The term “benzalkonium chloride” refers to an alkyldimethylbenzylammonium chloride of the general formula:

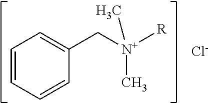

in which R represents an alkyl group of from 8 to 18 carbons. Generally, benzalkonium chloride is available as a mixture of the compounds, wherein R is a C₈₋₁₈ alkyl. Benzalkonium is also known as parasterol, alkyl benzyl dimethylammonium chloride, or alkyl dimethyl benzylammonium chloride.

The term “lower alkyl ketone” refers to a ketone having the general formula (R)(R′)C═O, wherein R and R′ are each independently selected from an alkyl. The term “alkyl” refers to a monovalent, saturated aliphatic hydrocarbon radical having the indicated number of carbon atoms. For example, a “C1-6 alkyl” or an “alkyl of 1-6 carbons” or “Alk 1-6” would refer to any alkyl group containing one to six carbons in the structure. “C1-20 alkyl” refers to any alkyl group having one to twenty carbons. Alkyl may be a straight chain (i.e. linear) or a branched chain. Lower alkyl refers to an alkyl of 1-6 carbons. Representative examples lower alkyl radicals include methyl, ethyl, n-propyl, n-butyl, n-pentyl, n-hexyl, isopropyl, isobutyl, isopentyl, amyl, sec-butyl, tert-butyl, tert-pentyl and the like. Higher alkyl refers to alkyls of seven carbons and above. These include n-heptyl, n-octyl, n-nonyl, n-decyl, n-dodecyl, n-tetradecyl, n-hexadecyl, n-octadecyl, n-eicosyl, and the like, along with branched variations thereof. The radical may be optionally substituted with substituents at positions that do not significantly interfere with the preparation of compounds falling within the scope of this invention and that do not significantly reduce the efficacy of the compounds. The alkyl may be optionally substituted with one to five substituents independently selected from the group consisting of halo, lower alkoxy, hydroxy, cyano, nitro, or amino. Preferred lower alkyl ketones include acetone, diethyl ketone and methyl ethyl ketone.

The term “dryness” refers to the state of skin having visible cracks or dry areas, or to the pain or sensation that can be associated with dry skin. The term “without dryness” means that the skin is in not appreciably more dry after sanitizing than before. Whether a composition promotes “dryness” or reduces “dryness” can be ascertained by studies that determine visually any dryness of the skin and the subjective symptoms reported by test subjects.

Other chemical terms are given their standard meaning as understood by one of skill in the art with guidance from standard texts and dictionaries.

### Compositions

While benzalkonium chloride and benzethonium chloride antimicrobial solutions are known in the art, they can require up to 30 wt. % alcohol (U.S. Pat. No. 7,112,559) as a drying agent. While alcohol is often added to the formulation as a drying agent to hasten evaporation of the solution from the skin, as mentioned above, the use of alcohol has drawbacks, such as drying of the skin. While emollients have been added to attempt to ameliorate this effect, the products can still cause dry and irritated skin. In light of this, benzalkonium chloride solutions that specifically do not contain alcohol have been attempted. While these products avoid the problems associated with alcohols, they also lack the functional benefits of the alcohol as set forth in U.S. Pat. No. 7,112,559 at column 3, lines 11-15.

Yet, purportedly, some quaternary compositions do not tend to have the severe skin-drying effects of alcohol-based sanitizers. See, U.S. Pat. No. 7,112,559, col. 1, 11. 42-49. However, despite reporting the purported drying effects of alcohol, the compositions in U.S. Pat. No. 7,112,559 will still contain up to 30% wt of an alcohol for the purpose of a solvent or a drying time enhancer. Id. at col. 3, 11. 12-15. Thus, these compositions teach that a substantial level of alcohol is required to be used as a drying agent.

Other compositions that avoid the use of alcohol altogether are known. The '559 patent discloses compositions that do not contain an alcohol or a drying agent. Accordingly, the art teaches that quaternary compositions contain alcohols as a necessary drying agent or the use of drying agents and alcohols is to be avoided entirely to prevent the accompanying skin drying affects attributed to the alcohols and drying agents. Another composition is described in U.S. Pat. No. 7,754,770 and is marketed under the brand name Nobac®. This composition purportedly contains one active ingredient (benzalkonium chloride) along with six inactive ingredients, and no alcohol. A foaming agent is required.

Accordingly, the suggested modifications to the existing art formulations are to use alcohols liberally, to add alcohols but only in amounts that are less than the antimicrobial amounts and the alcohols function as drying agents, or to formulate quaternary compositions without a drying agent or alcohol. None of these has been proven satisfactory. On the one hand, the sanitizing compositions must be wet on the skin for a sufficient time to effectively act as an antimicrobial. Ethanol has a drying time of about eight to thirteen seconds. Such drying times would not be expected to provide the effective killing necessary to reach the functionality of the compositions described herein. Significantly slower drying times, such as in the absence of drying agents, would lead to long-lasting residue on the skin. On the other hand, avoidance of drying agents altogether has been identified as an option that purportedly does not dry skin. It would be expected that the composition would remain wet for a significantly longer time than necessary. For industries that require frequent hand washing, such a medical facilities and food preparation, the longer and inconvenient period of drying would likely lead to less use and lower compliance, thus, defeating the purpose of providing the skin sanitizers. Additionally, as described elsewhere herein, wiping off the skin after sanitizing is not necessary as the compositions disclosed herein do not leave an appreciable or noticeable residue.

Thus, while it may be desirable to simply make the known compositions less drying to the skin by removing the alcohol or by adding an emollient that moisturizes the skin, the present inventors and the art as well have found that these solutions do not work in practice. There are several foreseeable and unforeseen problems with this approach. First, decreasing the ethanol content would also be expected to decrease the anti-microbial efficacy of the solution. The decrease in the amount of ethanol that would provide a substantially less-drying solution would also be expected to lower the efficacy to unacceptable, if not useless levels. Also seemingly straightforward is the removal of the drying agents altogether. Further, addition of a moisturizing agent to antimicrobial solutions has been accomplished. From a practical standpoint, however, the compositions with added moisturizer are not likely to dry on the skin. Rather, a tacky or greasy film would remain until wiped off. Foaming properties can also be diminished when moisturizer is present. All of these modifications would be expected to make formulating the composition more complicated and there would be no expectation that an effective and acceptable product in terms of both efficacy and appearance would be achievable.

However, the compositions disclosed herein overcome these problems. As set forth herein, it is the unique chemistries of the presently disclosed formulations that provide the desirable properties, in particular, with benzethonium chloride. Paradoxically, one of the aspects of the present disclosure that yields the desired products that are non-drying to the skin is the use of a lower alkyl ketone, such as acetone, which is also known to cause dry skin. However, acetone is not known to be as effective a drying agent as is ethanol. Additionally, acetone has a low vapor pressure and will evaporate quickly. Surprisingly, the present inventors have found an “oil effect” of the lower alkyl ketone component, in particular acetone, in combination, in particular, with a benzethonium chloride antimicrobial component, whereby the composition dries in about 15 to 20 seconds or less, e.g., 19, 18, 17, 16 seconds, preferably 16-20 seconds, does not leave a sticky residue and does not cause excessive dry skin like the dryness caused by alcohol containing sanitizers. Without being bound to any theory, it is believed that the quaternary ammonium salt results in slower evaporation of the lower alkyl ketone, specifically, acetone. This results in a drying time sufficient to be effective yet the compositions described herein do not cause the dryness to skin and the drying time is from about 15 seconds to 20 seconds or less. The compositions are therefore an exceedingly practical product for quick and repeated skin sanitization, in particular in the medical field and food industry, where effectiveness and timeliness are paramount. Specific amounts of acetone in combination with benzethonium chloride, and other components in the compositions, described elsewhere herein result in this advantageous and unexpected oil effect.

Additionally, the compositions described herein provide excellent efficacy and skin-feel characteristics with significantly fewer ingredients/chemistries than compositions in the art. The compositions described herein do not appreciably impact or remove normal skin oils, yet are effective antimicrobial compositions. Surprisingly and desirably, the compositions disclosed herein are clear solutions that are capable of foaming upon dispensing but do not require the addition of a separate foaming agent. In one embodiment, the subject matter described herein is directed to a concentrated sanitizing composition comprising: i. an antimicrobial component comprising a mixture of benzalkonium chloride, preferably benzethonium chloride as the sole quaternary ammonium salt, paraben and acetic acid in a ratio of about 1:0.8-1.3:1.6-2.5 (w:w:v), wherein the mixture comprises from about 0.00001% to about 0.1% (w/v); or from about 0.0002% to about 0.05% (w/v); or from about 0.03% to about 0.05% quaternary ammonium salt, e.g., benzalkonium chloride and/or benzethonium chloride (w/v), preferably benzethonium chloride as the sole quaternary ammonium salt; ii. a lower alkyl ketone; and iii. an emollient; and can further optionally contain an amount of water to dilute the solution to a viscosity appropriate for use a sanitizing solution, in particular, a skin sanitizing solution; and further optionally a fragrance.

The components above are listed as Food Additives or Generally Recognized As Safe (G.R.A.S.) by the F.D.A. or as minimum risk by the E.P.A. Thus, no wiping or rinsing off of the compositions is required. Many of the excipients described below are listed as such as well.

In embodiments, relatively low concentrations of disinfectant compounds are employed, so that the compositions can be produced economically. This is a desirable aspect as the compositions would be particularly useful in settings where sanitization is important yet not readily available, such as in underdeveloped areas and countries. In embodiments, each of the components of the sanitizing composition is considered non-toxic to mammals, resulting in no toxic components that need to be remediated as a result of the use of the composition. In addition, in certain embodiments, each of the components of the sanitizing solution is also G.R.A.S., thus resulting in even less environmental impact when disposing of used sanitizing solution. For example, in embodiments where the components of the compositions are G.R.A.S. or relatively inert, the compositions are readily degradable in the environment and can be used without concern of environmental build up.

Still further, the particular combination of components provides a stable composition which can withstand freezing and elevated temperatures. The compositions, therefore, are suitable for economical shipment. In embodiments, the compositions described herein can have an indefinite shelf life, for example, at least two years.

The sanitizing compositions described herein contain a minimum number of components. The antimicrobial component comprises three essential ingredients: quaternary ammonium salt, e.g., benzalkonium chloride and/or benzethonium chloride, preferably benzethonium chloride as the sole quaternary ammonium salt, paraben and acetic acid. In embodiments, the antimicrobial component consists essentially of a quaternary ammonium salt, such as benzalkonium chloride and/or benzethonium chloride, preferably benzethonium chloride as the sole quaternary ammonium salt, paraben and acetic acid. In these embodiments, the antimicrobial component will not contain an additional anti-microbial agent because it is not required due to the unique synergism of the three essential ingredients. The antimicrobial component is disclosed in U.S. Pat. No. 8,268,337, the contents of which are incorporated in its entirety by reference. The ratio of each ingredient in the antimicrobial component is relative to the other ingredients in the antimicrobial component, which is understood to be dispersed into the end-use skin sanitizing composition. In each embodiment, it is preferred that the quaternary ammonium salt is benzethonium chloride. Benzethonium chloride is desirably a FDA approved topical disinfectant.

One embodiment of the present invention provides a sanitizing solution composition comprising, an antimicrobial mixture comprising a mixture of benzalkonium chloride and/or benzethonium chloride, preferably benzethonium chloride as the sole quaternary ammonium salt, paraben and acetic acid in a ratio of 1:0.8-1.3:1.6-2.5 (w:w:v) (the mixture is also referred to herein as the “antimicrobial component” and “FNC”); a lower alkyl ketone; and an emollient. These ingredients are present in the solution in relative concentrations. Each component can be described relative to the amount of FNC whereby the sum of all components is 100% and all specific amounts and ranges are encompassed by the following relative amounts. For one part FNC, there is from about 0.5 to about 2 parts carrier; For one part FNC, there is from about 0.7 to about 1.5 parts carrier; For one part FNC, there is from about 0.8 to about 1.2 parts carrier, or, in embodiments, about 1 part carrier, e.g., water. For one part FNC, there is from about 0.01 to about 0.33 parts lower alkyl ketone; For one part FNC, there is from about 0.1 to about 0.2 parts lower alkyl ketone; For one part FNC, there is from about 0.14 to about 0.18 parts lower alkyl ketone, or, in embodiments, about 0.16 part lower alkyl ketone, e.g., acetone. For one part FNC, there is from about 0.5 to about 4 parts emollient; for one part FNC, there is from about 1.0 to about 3.0 parts emollient; for one part FNC, there is from about 1.5 to about 2.5 parts emollient, or, in embodiments, about 2 parts emollient, e.g., aloe vera. For one part FNC, when present, there is from about 0.001 to about 0.010 parts fragrance; for one part FNC, there is from about 0.01 to about 0.1 parts fragrance; for one part FNC, there is from about 0.01 to about 0.03 parts fragrance, or, in embodiments, about 0.018 part fragrance, e.g., lavender.

Particularly useful compositions contain each of the following components in amounts relative to FNC: For one part FNC, from about 0.7 to about 1.5 parts carrier; For one part FNC; For one part FNC, there is from about 0.1 to about 0.2 parts lower alkyl ketone; For one part FNC, there is from about 1.5 to about 2.5 parts emollient; For one part FNC, there is from about 0.01 to about 0.03 parts fragrance.

In some embodiments, other particularly useful compositions contain each of the following components in amounts relative to the lower alkyl ketone: For one part lower alkyl ketone, there is from about 2 parts to about 25 parts FNC; from about 4 parts to about 20 parts emollient; and from about 0.001 to about 0.0010 parts fragrance. Preferably, for one part lower alkyl ketone, there is from about 8 parts to about 16 parts FNC; from about 8 parts to about 16 parts emollient; and from about 0.001 to about 0.0010 parts fragrance. More preferably, for one part lower alkyl ketone, there is from about 10 parts to about 14 parts FNC; from about 10 parts to about 14 parts emollient; and from about 0.001 to about 0.0010 parts fragrance. Most preferably, for one part lower alkyl ketone, there is about 12 parts FNC; about 12 parts emollient; and from about 0.001 to about 0.0010 parts fragrance. In all of these embodiments, a useful amount of fragrance is about 0.004 parts.

More useful compositions contain amounts of components that result in an oil effect and that additionally provide the desired physical properties of the compositions. For example, when benzethonium chloride is the sole quaternary ammonium salt, the compositions additionally have the desired foaming properties not found with benzalkonium chloride or when there is an absence of foaming agents. However, the addition of foaming agents increase production costs and would further require that the agents work with all the other components. Accordingly, in useful embodiments, the compositions can comprise excipients but do not contain a foaming agent. Foaming agents are common ingredients and are well-known to formulators in this field. In embodiments, the foaming agent is G.R.A.S.

In embodiments, incorporation of a trihydric alcohol is avoided. In embodiments, incorporation of glycerol is avoided. That is, the compositions do not contain these materials.

One embodiment of the present invention provides a concentrated sanitizing solution composition comprising a mixture of quaternary ammonium salt, such as benzalkonium chloride and/or benzethonium chloride, preferably benzethonium chloride as the sole antimicrobial agent, paraben and acetic acid in a ratio of 1:0.8-1.3:1.6-2.5 (w:w:v); a lower alkyl ketone; and an emollient, wherein the concentrated solution is later diluted from about 10-fold to about 10,000-fold prior to use. A preferred aspect of this embodiment is a concentrated sanitizing solution composition comprising about 0.5% benzethonium chloride (w/v), 0.5% paraben (w/v), and about 1% acetic acid (v/v), wherein the concentrated solution is diluted from about 10-fold to about 10,000-fold prior to use.

It is also possible to provide the concentrate not as a solution but in solid form or even a viscous liquid form having a lower volume for ease of shipping. Examples include a pre-formed mixture of solids or powders or as a freeze-dried remnant of a solution. For example, acetic acid in a solid or powdered form that dissolves upon addition of water. Alternatively, acetic acid can be provided as glacial acetic acid or in another concentrated liquid form in a separate container, with the remaining components being provided as solids (their normal purified forms). When acetic acid is provided as a solid, the amount to be used can still be expressed as a volume equivalent and is done so for simplicity in this specification. For example, a preferred ratio of components provides benzethonium chloride, paraben and acetic acid in a ratio of 1:0.8-1.3:1.6-2.5 (w:w:v) in the form of a concentrate. When a solid composition is provided with acetic acid itself being provided in the form of a solid, enough of the solid form of acetic acid is provided so that the appropriate equivalent volume of acetic acid (1.6-2.5 ml of acetic acid per gram of benzethonium chloride) is available when the components of the mixture are dissolved.

Some of the other components of the compositions can also be provided as solids in individual containers for increased stability during shipment and/or storage. Alternatively, individual components can be pre-dissolved and provided in the form of concentrated solutions for ease of later mixture and further dilution. When individual components are provided for later mixture and/or dilution, the individual components are generally shipped in a common outer container along with instructions for their proper mixing to form the concentrate composition and/or any of its diluted forms. Provision of instructions that describe preparation of compositions of the invention from commercially available supplies of individual components are considered to be equivalent to provision of the mixtures as described herein.

The concentrate composition which is diluted to a “use solution” prior to its utilization as a sanitizer is beneficial primarily for reasons of economics of shipping. The concentrate would normally be diluted with water or an aqueous diluent to form a use solution. The general constituent concentrations of the sanitizing concentrate formulated in accordance with the compositions described herein are found in Table 1:

TABLE 1

| Constituent | Range A Amount (%) | Range B Amount (%) | Target Amount (%) |
|---|---|---|---|
| Benzalkonium | 0.2-2.5 | 0.3-1.5 | 0.5 |
| chloride (w/v) and/or | | | |
| Benzethonium | | | |
| chloride (w/v) | | | |
| Paraben (w/v) | 0.2-2.5 | 0.3-1.5 | 0.5 |
| Acetic acid (w/v) | 0.4-5.0 | 0.6-3.0 | 1.0 |

Other ingredients can be admixed with those above in the following amounts listed in Table 2:

TABLE 2

| Component | Amount | |
|---|---|---|
| Concentrate Composition | 55 | gal |
| Water | 55 | gal |
| Aloe | 110 | gal |
| Acetone | 9 | gal |
| Fragrance (optional) | 1 | gal |

Although the concentrate or solid precursor mixture is normally intended for use with added water alone in order to simplify preparation of the final use solution by potentially unskilled workers, it is also possible to use other aqueous compositions as a diluting solvent. For example, small amounts of a salt (e.g., table salt, to provide a desired ionic strength) could be added in an initial dilution to modify solubility of individual components of the mixture. Preferred aqueous compositions used for diluting from solids or concentrates contain less than 10% non-water components (by weight), more preferably less than 5%, even more preferably less than 2%, and most preferably contain only water (along with any trace components as might be present in a municipal water supply or other source of drinking-quality water).

When dealing with a concentrate composition, the level of active components in a concentrate composition is dependent on the intended dilution factor. Generally, a dilution of about 1 fluid ounce to about 1.0 to about 150.0 gallons of water is used for aqueous antimicrobial sanitizing solutions. The composition shown in Table 1 above would be useful in a range from about 0.8 fluid ounce per gallon water to about 1.6 fluid ounce per gallon of water.

The pH of the composition can be any pH at which the composition retains its stability and/or rheological properties. One of ordinary skill in the art would know the appropriate pHs which are suitable for the composition. Particularly useful pHs of the composition are from about 0.2 to about 10. Specifically, the pH can be from about 3 to below 7, e.g., a pH of about 6.

Water is included as a diluent in an amount sufficient to make the total composition 100% by weight. The water may be tap water, but is preferably distilled and/or deionized water. If the water is tap water, it is preferably appropriately filtered in order to remove any undesirable impurities such as organics or inorganics, especially minerals salts which are present in hard water and which may thus interfere with the operation of the other components of the composition, as well as any other optional components of the composition. Water is added in amounts which are sufficient to form the diluted composition which amount is prescribed to be useful for antimicrobial efficacy and the application by the end-user such as a hand wash and the like. Water is typically used as a filler solvent for “spray on” or “light duty” compositions or to make up the balance of concentrates. The compositions, other than concentrates wherein the consumer adds the carrier prior to use, comprise at least 20%, preferably, at least 25%, more preferably at least 30%, 35% or 40% by weight, of water as carrier. Concentrates can be diluted prior to use to the aforementioned concentrations. The concentrate which can be diluted with a carrier will comprise quaternary ammonium salt, e.g., benzalkonium chloride and/or benzethonium chloride, preferably benzethonium chloride as the sole antimicrobial agent, paraben and acetic acid in a ratio of about 1:0.8-1.3:1.6-2.5; a lower alkyl ketone; and an emollient.

While water is preferred, other known carriers may be used so long as the efficacy and drying time is not affected. The carrier makes up a large portion of the compositions may essentially be the balance of the composition apart from the active components. The carrier concentration and type will depend upon the nature of the composition as a whole, the environmental storage and method of application including concentration of the antimicrobial agent, among other factors. Notably the carrier should be chosen and used at a concentration which does not inhibit the antimicrobial efficacy and the drying time of the compositions.

The fragrance can be an essential oil based fragrance. Particularly, the essential oil based fragrance is about 0.01% to about 0.5% by weight, suitably about 0.05% to about 0.35% by weight. Representative examples of an essential oil based fragrance which may be compatible with the cleaning and disinfecting composition of the present invention include clove oil, lavender oil and citrus oil. In an embodiment, the citrus essential oils may be selected from lavender, citrus, sandalwood, gardenia, jasmine, mint, wintergreen, rosemary, vanilla, coconut, lilly of the valley, frankincense, and/or myrrh. Others include orange, grapefruit, lime, lemon, lemongrass, blood orange, petitgrain and litsea cubeba. In a further embodiment, the citrus essential oil is litsea cubeba.

In an embodiment, the emollient may be aloe vera, acetone, and fragrance or other hydrolyzed proteins having emollient properties. The emollient must not interact with the “oil effect” and compromise the antimicrobial effectiveness and drying time of the composition. In a particular embodiment, aloe vera has been shown to work with the oil effect of benzethonium chloride and acetone.

The compositions described herein may be formulated to be dispersed from a ready-to-use dispenser system. For instance, the cleaning and disinfecting compositions may be dispelled from a trigger or finger pump bottle, a squeeze bottle or a pressurized sprayer to produce a foam. For instance, the compositions may be dispensed from a trigger or finger pump bottle, a squeeze bottle, pressurized canister or a wall dispenser actuated by a motion detector.

### Optional Excipients and Adjuvants

Preferred embodiments consist essentially of the components described in Table 2. Additional inert ingredients, such as fillers, when added in non-functional amounts, do not materially change the compositions. While not necessary for product performance as a skin sanitizer having improved dermatological effects, in embodiments, the compositions may also optionally include any number of adjuvants which add beneficial properties of stability, sequestration, coating and rinsing, etc. These adjuvants may be preformulated with the sanitizing agent of the invention or added to the system simultaneously, or even after, the addition of the sanitizing agent of the invention. Such adjuvants include components provide moisturizing, healing, and/or synergistic properties.

While not required, a buffer may be included in the composition to maintain a relatively constant pH. The type of buffer is not essential as any known buffer, which is compatible with the components of the composition, may be used. For example, the buffer may be a solution of a weak acid and its salt or a solution of a weak base and its salt. In an embodiment of the present invention, the buffer is about 0.06% to about 1.25% by weight, suitably about 0.25% to about 0.75% by weight. In yet another embodiment of the invention, the buffer is a combination of citric acid and sodium citrate. In a more specific embodiment of the invention, the citric acid is present in an amount of about 0.01% to about 0.25% by weight and the sodium citrate is present in an amount of about 0.05% to about 1% by weight.

One or more other ingredients may optionally be included in the compositions to increase aesthetic or other beneficial properties. Such optional ingredients may include deodorizers, coloring agents, descaling compounds, co-surfactants and the like. These additional ingredients, however, must be compatible with the other core components of the compositions and, as discussed above, avoid negatively affecting the environmental as well as the health and safety profiles of the compositions.

The optional adjunct ingredients can be included in compositions at their conventional art-established levels for use (generally, adjunct materials comprise, in total, from about 30% to about 99.9%, preferably from about 70% to about 95%, by weight of the compositions). Examples of adjunct ingredients are buffers, builders, chelants, filler salts, dispersants, enzymes, enzyme boosters, perfumes, thickeners, clays, solvents, and mixtures thereof. This list is not meant to be totally inclusive or exclusive of materials that are compatible.

Various organic compounds can be included which facilitate the functions provided above. While avoiding simple alkyl alcohols such as ethanol, isopropanol, n-propanol, and the like that provide anti-microbial properties but are drying to the skin, polyols can be useful additives. These include propylene glycol, polyethylene glycol, sorbitol, and the like. Any of these compounds may be used singly or in combination with other organic or inorganic constituents or, in combination with water or in mixtures thereof.

Thickeners useful in the present compositions are those which do not leave contaminating residue on the skin. Generally, thickeners that may be used in the present invention include natural gums such as xanthan gum. Also useful in the present invention are cellulosic polymers, such as carboxymethyl cellulose. Generally, the concentration of thickener will be dictated by the desired viscosity within the final composition. However, as a general guideline, concentration of thickener within the present composition ranges from about 0.1 wt-% to about 1.5 wt-%, preferably from about 0.1 wt-% to about 1.0 wt-%, and most preferably from about 0.1 wt-% to about 0.5 wt-%.

Antibiotics, antivirals, or other antimicrobial agents may optionally be incorporated in either or both of the first part and the second part. Suitable agents will be well known to those of ordinary skill in the art. Examples include cationics, amphoterics and phenolics. Humectants, moisturizers and fragrances may optionally be included in the first part or (preferably) the second part, as is well known in the art. Corrosion inhibitors may be included in the first part and/or the second part, for improved packaging and protection of the dispenser.

### Articles of Manufacture

In an embodiment, the subject matter described herein is a sachet, packet etc. that contains the sanitizing solution in a convenient package for use in a dispensing device. This device can be a spray pump or foaming pump type dispenser whereby the user presses a button or pushes a lever to dispense the composition or a motion actuated dispenser. Towelettes, e.g., a fabric dimensioned for swiping, pre-moistened and comprising the compositions described herein are also disclosed.

In one embodiment, the subject matter described herein is directed to a package containing a single use article of a concentrated sanitizing composition comprising: i. an antimicrobial component comprising a mixture of quaternary ammonium salt, e.g., benzalkonium chloride and/or benzethonium chloride, preferably benzethonium chloride as the sole antimicrobial agent, paraben and acetic acid in a ratio of about 1:0.8-1.3:1.6-2.5 (w:w:v), wherein the mixture comprises from about 0.00001% to about 0.1% (w/v); or from about 0.0002% to about 0.05% (w/v); or from about 0.03% to about 0.05% benzalkonium chloride and/or benzethonium chloride (w/v), preferably benzethonium chloride as the sole antimicrobial agent; ii. a lower alkyl ketone; and iii. an emollient; and optionally a fragrance. In this embodiment, the compositions described herein may also be incorporated into a towelette form. The towelettes may be packaged individually or in bulk for individual distribution.

Further, the cleaning and disinfecting compositions of the present invention may be incorporated into other formulations or carriers having antimicrobial or disinfecting properties. These formulations may be those of antiseptics, soaps or lotions.

### Methods of Sanitizing

In one embodiment, the subject matter described herein is directed to methods of sanitizing skin by contacting the area of skin to be sanitized with a concentrated sanitizing composition comprising: i. an antimicrobial component comprising a mixture of quaternary ammonium salt, e.g., benzethonium chloride, paraben and acetic acid in a ratio of about 1:0.8-1.3:1.6-2.5 (w:w:v), wherein the mixture comprises from about 0.00001% to about 0.1% (w/v); or from about 0.0002% to about 0.05% (w/v); or from about 0.03% to about 0.05% quaternary ammonium salt (w/v), preferably benzethonium chloride as the sole antimicrobial agent; ii. a lower alkyl ketone; and iii. an emollient, wherein the skin is sanitized. In this embodiment, the contacting lasts 20 seconds or less before the composition is dry to the touch. The methods specifically do not require wiping off any residue as there is essentially none. As used herein, contacting includes rubbing, spreading, etc. the composition across the skin to be sanitized. After the composition dries on the skin, the skin is sanitized.

The subject matter described herein is directed to the following specific embodiments, which include:
1. A skin sanitizing composition comprising:
   i. an antimicrobial component consisting essentially of a quaternary ammonium compound, paraben and acetic acid in a ratio of 1:0.8-1.3:1.6-2.5 (w:w:v);
   ii. an emollient; and
   iii. a lower alkyl ketone.
2. The composition of embodiment 1, further comprising a quantity of water.
3. The composition of embodiment 1 or 2, further comprising a fragrance.
4. The composition of embodiment 1, 2 or 3, wherein the quaternary ammonium compound is benzethonium chloride.
5. The composition of embodiment 1, 2, 3 or 4, wherein the emollient comprises a hydrolyzed protein.
6. The composition of embodiment 1, 2, 3, 4 or 5, wherein the emollient is aloe vera.
7. The composition of embodiment 1, 2, 3, 4, 5 or 6, wherein the lower alkyl ketone is acetone.
8. The composition of embodiment 1, 2, 3, 4, 5, 6 or 7, wherein said emollient is present in an amount of about 3 to 5 lbs per gallon of the composition.
9. The composition of embodiment 1, 2, 3, 4, 5, 6, 7 or 8, wherein said lower alkyl ketone is present in an amount of about 0.002 to 0.2 lbs per gallon of the composition.
10. The composition of embodiment 1, 2, 3, 4, 5, 6, 7, 8 or 9, wherein said composition dries about 10 seconds to about 20 seconds or less after initial contact with skin.
11. The composition of embodiment 1, 2, 3, 4, 5, 6, 7, 8, 9 or 10, wherein said composition dries in about 15 seconds to about 20 seconds or less after initial contact with skin.
12. The composition of embodiment 1, 2, 3, 4, 5, 6, 7, 8, 9, 10 or 11, wherein said composition is a foam when contacted with skin.
13. A skin sanitizing composition comprising:
   i. an antimicrobial component consisting essentially of a quaternary ammonium compound, paraben and acetic acid in a ratio of 1:0.8-1.3:1.6-2.5 (w:w:v);
   ii. aloe vera, in an amount of about 3 to 5 lbs per gallon of the composition; and
   iii. acetone in an amount of about 0.002 to about 0.2 lbs per gallon of the composition.
14. The composition of embodiment 13, wherein said aloe vera is present in an amount of about 4.2 lbs per gallon, and said acetone is present in an amount of about 0.02 lbs per gallon.
15. The composition of embodiment 13 or 14, further comprising water.
16. The composition of embodiment 13, 14 or 15, further comprising a fragrance.
17. A skin sanitizing composition comprising:
   i. an antimicrobial component consisting essentially of benzethonium chloride, paraben and acetic acid in an amount of about 0.04 lbs per gallon of the composition and at a ratio of 1:0.8-1.3:1.6-2.5 (w:w:v);
   ii. aloe vera, in an amount of about 4.2 lbs per gallon of the composition;
   iii. acetone in an amount of about 0.02 pounds per gallon of the composition;
   iv. water in an amount of about 4.05 lbs per gallon of the composition; and
   v. fragrance in an amount of about 0.002 lbs per gallon of the composition.

A particular formulation comprises:
i. an antimicrobial component consisting essentially of benzethonium chloride, paraben and acetic acid in an amount of about 1: 0.04 and at a ratio of 1:0.8-1.3:1.6-2.5 (w:w:v);
ii. aloe vera, in an amount of about 1:4.2;
iii. acetone in an amount of about 1:0.02;
iv. water in an amount of about 1:2; and
v. fragrance in an amount of about 1:0.002.
18. A method of sanitizing skin comprising, contacting the skin sanitizing composition of embodiments 1-17 with skin for an effective amount of time.
19. The method of embodiment 18, wherein said time is about 15 seconds to about 20 seconds or less.
20. The method of embodiment 18 or 19, wherein the method does not comprise wiping off the composition from the skin after sanitization.
21. A method of preparing a skin sanitizing solution comprising, contacting an antimicrobial component consisting essentially of a quaternary ammonium salt, paraben and acetic acid in a ratio of 1:0.8-1.3:1.6-2.5 (w:w:v) with an emollient and a lower alkyl ketone, and optionally a fragrance, wherein said components can be added in any order.
22. The method of embodiment 21, wherein the quaternary ammonium salt is benzethonium chloride.
23. A single use article comprising: i. an antimicrobial component consisting essentially of a quaternary ammonium salt, paraben and acetic acid in a ratio of 1:0.8-1.3:1.6-2.5 (w:w:v); ii. an emollient; and iii. a lower alkyl ketone; and optionally, iv. a fragrance.
24. The article of embodiment 23, wherein said article is a swab or towelette.
25. A bulk solution for sanitizing the skin comprising,

| Component | Amount | |
|---|---|---|
| Concentrate Composition | 1 | part |
| Water | 1 | part |
| Aloe | 2 | parts |
| Acetone | 0.16 | part |
| Fragrance | 0.018 | part |

wherein said bulk solution is a volume of at least one pint, or one quart, or one gallon, or more than 50 gallons.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

The following examples are offered by way of illustration and not by way of limitation.

### EXAMPLES

### Example 1

### Representative Formulations are shown in Table 3.

TABLE 3

| Mix | Components and amounts | Product characteristics |
|---|---|---|
| 228 | 1 part FNC | Prolonged wetness; |
| | 11 parts Aloe | Sticky |
| | 6 Dp-PEP | |
| 231-B | 1 part FNC | Watery; |
| | 3 parts Aloe | Low foam |
| | 6 Dp-LAV | |
| | 1/8 part MgSO₄•7H₂O | |
| 234 | 1 part FNC | Prolonged wetness |
| | 3 parts Aloe | |
| | 2 Dp-PEP | |
| 231-D | 1 part FNC | Foams well; |
| | 1 part Aloe | No prolonged |
| | 3 Dp-PEP | wetness |
| | ¼ part Acetone | |
| 229 | 1 part BAC (19.2 | Low or no foam |
| | concentrate) | |
| | 3 parts Aloe | |
| | No fragrance | |
| | ½ part MgSO₄•7H₂O | |
| 228-A | 3 parts FNC (10%) | Low foam; |
| | 3 parts Aloe | Dries quickly |
| | 1 DP-PEP | |
| | 1/3 part MgSO₄•7H₂O | |
| 231-C | 5 parts FNC (5%) | Thin viscosity; |
| | 1 part Aloe | Prolonged wetness |
| | 10 mls LAV | |
| 231-A | 1 part FNC (10%) | No foam; |
| | 3 parts Aloe | Watery |
| | 1 DP-PEP | |
| | 5 parts MgSO₄•7H₂O | |
| 253 | 1 part FNC (10%) | Low foam; |
| | 5 parts Aloe | Prolonged wetness |
| | 1 Dp-PEP | |
| 228-B | 1 part FNC | Exhibited increased |
| | 8 parts Aloe | wetness |
| | 1 ml LAV | |
| 228-C | 2 parts FNC | Acceptable foam; |
| | 8 parts Aloe | Prolonged wetness |
| | ½ ml LAV | |
| 228-D | 1 part FNC (2%) | Prolonged wetness; |
| | 8 parts Aloe | Watery |
| | ½ ml LAV | |
| 228-E | 1 part FNC (concentrate) | Watery; |
| | 8 parts Aloe | Sticky |
| | ½ ml LAV | |
| 228-F | 1 oz + 1 oz BW | Acceptable foam; |
| | 7 oz Aloe | Sticky |
| | ½ ml LAV | |
| 231-C | 3 oz FNC | Sticky; |
| | 3 oz Aloe | Prolonged wetness |
| | 5 DP LAV | |
| | ¼ oz Acetone | |
| | ¼ oz glycerin | |
| 231-E | 1 oz FNC (10%) | Acceptable foaming; |
| | 3 oz Aloe | Prolonged wetness |
| | 1 Dp-PEP | |
| 231-F | 1 oz FNC | Low foam; |
| | 5 oz Aloe | Quick drying |
| | 6 Dp-PEP | |
| | ¼ oz Acetone | |
| 228-F | 2 oz FNC + 2 oz BAC | Acceptable foam; |
| | 4 oz Aloe | Sticky |
| | ½ ml LAV | |
| 282 | 3 oz FNC | Foaming; |
| | 3 oz Aloe | Approx. 20 secs |
| | ¼ oz Acetone | drying time |
| | ½ ml PEP | |

“FNC” refers to antimicrobial component consisting essentially of benzalkonium chloride, paraben and acetic acid in a ratio of 1:0.8-1.3:1.6-2.5 (w:w:v); Dp = drop; PEP = peppermint

### Example 2

Testing was performed per the Handbook of Topical Antimicrobials by Daryl S. Paulson. Desirable results for microbicidal are provided in the Table 4 below.

TABLE 4

| | EXPOSURE | % |
|---|---|---|
| ORGANISM | TIME | REDUCTION |
| Campylobacter | 15 sec | 99.9999 |
| Candida albicans | 15 sec | 99.9889 |
| Enterococcus (VRE) | 15 sec | 99.9997 |
| Escherichia coli | 15 sec | 99.9999 |
| Klebsiella pneumonia | 15 sec | 99.9992 |
| Listeria monnocytogens | 15 sec | 99.9990 |
| Pseudomonas aeruginosa | 15 sec | 99.9993 |
| Salmonella typhi | 15 sec | 99.9999 |
| Staphylococcus aureus | 15 sec | 99.9999 |
| Staphylococcus (MRSA) | 15 sec | 99.9993 |
| Streptococcus pneumonia | 15 sec | 99.9999 |

### Example 3

### Dermal Studies

Medical and surgical personnel sanitized hands more than 15 times per day with a formulation as described in embodiment 17. Personnel then reported on the condition of skin after five days of use of the composition. Trial participants provided assessment of three criteria—visible and sensation of dryness, visible cracking, and tingling sensation. While use of a non-drying product can decrease the level of dryness by a measurable amount as compared to known products, some symptoms of dryness are expected to persist. With frequent use, even a non-drying formula in the art would be expected to produce dryness. Yet, the compositions described herein produced no visible cracking or drying on the skin of the study participants. Therefore, it is unexpected that there was no dryness, cracking or tingling reported in the study group. Even more surprising are these findings given the frequency with which personnel used the product over a period of consecutive days that lasted a full work-week.
i. Field Trial—A
Location: Medical office practice
Trial Participants: 10 medical staff
Current level of hand sanitizations: each personnel foams hands >15 times, per day

Comparative baseline: 6 personnel report skin problems, such as dryness and tingling with use of current hand disinfectant product containing alcohol.
Trial Protocol:
   1. Exclusive use of formulation for hand disinfectant >15 times per day
   2. Duration: 5 days
   3. Participants to report dermal and non-dermal symptoms
Results:

Dermal assessment: Each participant reported soft, crack-free hand skin after use of formulation on hands.

Non-dermal assessment: “clean fragrance,” no alcohol odor, after use of formulation on hands
ii. Field Trial—B
Location: Medical office practice
Trial Participants: 3 medical staff
Current level of hand sanitizations: each personnel foams hands >10 times, per day

Comparative baseline: All personnel report skin problems, such as dryness, chapped skin, and unpleasant odor of commercial sanitizing foam product.
Trial Protocol:
   1. Exclusive use of formulation for hand disinfectant >10 times per day
   2. Duration: 5 days
   3. Participants to report dermal and non-dermal symptoms
Results:

Dermal assessment: Each participant reported healthy, soft hands even with repeated, daily use of formulation on hands.

Non-dermal assessment: pleasant fragrance, no alcohol odor, after use of formulation on hands
iii. Field Trial—C
Location: Surgical practice
Trial Participants: 3 operating room staff

Current level of hand sanitizations: “foam-in, foam-out” operating room procedure; each personnel foams hands >15 times, per day

Comparative baseline: All personnel report skin problems, such as visibly dry and cracked skin, and unpleasant odor of commercial sanitizing foam product.
Trial Protocol:
   1. Exclusive use of formulation for hand disinfectant >35 times per day
   2. Duration: 5 days
   3. Participants to report dermal and non-dermal symptoms
Results:

Dermal assessment: Each participant reported significant improvement in skin condition, even with 35 uses of formulation on hands per day.

Non-dermal assessment: pleasant, clean fragrance, much improved product compared to commercially available product.

All technical and scientific terms used herein have the same meaning. Efforts have been made to ensure accuracy with respect to numbers used (e.g. amounts, temperature, etc.) but some experimental errors and deviations should be accounted for.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

The term “about” preferably indicates a difference between the actual measurement and the intended measurement of no more than 20% when a process measurement actually used is compared to the most accurate measurement available for the type of measurement being made, more preferably a difference of no more than 10%, even more preferably no more than 5%, and most preferably that the difference would fall within two standard deviations from the mean of a (typically commercially available) measuring instrument being used in the process making the measurement, the latter “within-two-standard-deviations” standard typically being a measure of equality. Differences deliberately created by infringers in an attempt to avoid the scope of the claims are also covered by the term “about” as long as the differences remain in the range of equivalents for the invention. All of the preferences as recited above for accidental differences also apply to deliberate differences created by infringers (i.e., a deliberate infringer who is 20% off from a stated range is within a preferred range of equivalents, and so forth). If the word “about” is not present when referring to a measurable quantity, the factors relating to accidental- and deliberate-measurement differences as discussed above are understood to apply. As used herein, the term “about,” when referring to a value is meant to encompass variations of, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Where a range of values is provided, it is understood that each intervening value, to the tenth of the unit of the lower limit, unless the context clearly dictates otherwise, between the upper and lower limit of the range and any other stated or intervening value in that stated range, is encompassed within the invention. The upper and lower limits of these small ranges which may independently be included in the smaller rangers is also encompassed within the invention, subject to any specifically excluded limit in the stated range. Where the stated range includes one or both of the limits, ranges excluding either or both of those included limits are also included in the invention.

1. A skin sanitizing composition comprising:
i. an antimicrobial component consisting essentially of a quaternary ammonium compound, paraben and acetic acid in a ratio of 1:0.8-1.3:1.6-2.5 (w:w:v);
ii. an emollient; and
iii. a lower alkyl ketone.

2. The composition of claim 1, further comprising a quantity of water.

3. The composition of claim 1, further comprising a fragrance.

4. The composition of claim 1, wherein the quaternary ammonium compound is benzethonium chloride.

5. The composition of claim 1, wherein the emollient comprises a hydrolyzed protein.

6. The composition of claim 1, wherein the emollient is aloe vera.

7. The composition of claim 1, wherein the lower alkyl ketone is acetone.

8. The composition of claim 1, wherein said emollient is present in an amount of about 3 to 5 lbs per gallon of the composition.

9. The composition of claim 1, wherein said lower alkyl ketone is present in an amount of about 0.002 to 0.2 lbs per gallon of the composition.

10. The composition of claim 1, wherein said composition dries about 15 seconds to about 20 seconds or less after initial contact with skin.

11. The composition of claim 10, wherein said composition dries about 16 seconds to about 20 seconds or less after initial contact with skin.

12. The composition of claim 1, wherein said composition is a foam when contacted with skin.

13. A skin sanitizing composition comprising:
i. an antimicrobial component consisting essentially of a quaternary ammonium compound, paraben and acetic acid in a ratio of 1:0.8-1.3:1.6-2.5 (w:w:v);
ii. aloe vera, in an amount of about 3 to 5 lbs per gallon of the composition; and
iii. acetone in an amount of about 0.002 to about 0.2 lbs per gallon of the composition.

14. The composition of claim 13, wherein said aloe vera is present in an amount of about 4.2 lbs per gallon, and said acetone is present in an amount of about 0.02 lbs per gallon.

15. The composition of claim 13, further comprising water.

16. The composition of claim 13, further comprising a fragrance.

17. A skin sanitizing composition comprising:
i. an antimicrobial component consisting essentially of benzethonium chloride, paraben and acetic acid in an amount of about 0.04 lbs per gallon of the composition and at a ratio of 1:0.8-1.3:1.6-2.5 (w:w:v);
ii. aloe vera, in an amount of about 4.2 lbs per gallon of the composition;
iii. acetone in an amount of about 0.02 pounds per gallon of the composition;
iv. water in an amount of about 4.05 lbs per gallon of the composition; and
v. fragrance in an amount of about 0.002 lbs per gallon of the composition.

18. A method of sanitizing skin comprising, contacting the skin sanitizing composition of claim 1 with skin for an effective amount of time.

19. The method of claim 18, wherein said time is about 15 seconds to about 20 seconds or less.

20. The method of claim 18, wherein the method does not comprise wiping off the composition from the skin after sanitization.

21. A method of preparing a skin sanitizing solution comprising, contacting an antimicrobial component consisting essentially of a quaternary ammonium salt, paraben and acetic acid in a ratio of 1:0.8-1.3:1.6-2.5 (w:w:v) with an emollient and a lower alkyl ketone, and optionally a fragrance, wherein said components can be added in any order.

22. The method of claim 21, wherein the quaternary ammonium salt is benzethonium chloride.

23. A single use article comprising: i. an antimicrobial component consisting essentially of a quaternary ammonium salt, paraben and acetic acid in a ratio of 1:0.8-1.3:1.6-2.5 (w:w:v); ii. an emollient; and iii. a lower alkyl ketone; and optionally, iv. a fragrance.

24. The article of claim 23, wherein said article is a swab or towelette.

25. A bulk solution for sanitizing the skin comprising,

| Component | Amount | |
|---|---|---|
| Concentrate Composition | 1 | part |
| Water | 1 | part |
| Aloe | 2 | parts |
| Acetone | 0.16 | part |
| Fragrance | 0.018 | part |

wherein said bulk solution is a volume of at least one pint, or one quart, or one gallon, or more than 50 gallons.
